(19) United States
(12) Patent Application Publication
(10) Pub. No.: US 2004/0171063 A1
(43) Pub. Date: Sep. 2, 2004

(54) Local descriptors of protein structure

(75) Inventors: Krzysztof A. Fidelis, Brentwood, CA (US); Andriy A. Kryshtafovych, Livermore, CA (US)

Correspondence Address: Eddie E. Scott, Assistant Laboratory Counsel, Lawrence Livermore National Laboratory, P.O. Box 808, L-703, Livermore, CA, 94551, US

(73) Assignee: The Regents of the University of California

(21) Appl. No.: 10/378,492

(22) Filed: Feb. 27, 2003

(51) Int. Cl. G01N 33/53; G06F 19/00; G01N 33/48; G01N 33/50

(52) U.S. Cl. 435/7.1; 702/19

(57) ABSTRACT

Information about a protein is produced by a number of steps including: Developing a specification of how local descriptors of protein structure are defined. Developing a local descriptors of protein structure database including calculated descriptors. Developing a definition of inter-descriptor similarity measure. Developing a local descriptors of protein structure class database of descriptors organized by their similarity. Producing information about any protein by analyzing said protein using the local descriptors of protein structure class database of descriptors organized by their similarity.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to obtaining information about protein structure and more particularly to local descriptors of protein structure.

2. State of Technology

International Patent Application No. WO 93/01484 published Jan. 21, 1993 for a method to identify protein sequences that fold into a known three-dimensional structure by David Eisenberg et al. assigned to the Regents of the University of California provides the following background information, ” A computer-assisted method for identifying protein sequences that fold into a known three-dimensional structure. The inventive method attacks the inverse protein folding problem by finding target sequences that are most compatible with profiles representing the structural environments of the residues in known three-dimensional protein structures. The method starts with a known three-dimensional protein structure and determines three key features of each residue's environment within the structure: (1) the total area of the residue's side-chain that is buried by other protein atoms, inaccessible to solvent; (2) the fraction of the side-chain area that is covered by polar atoms (O, N) or water, and (3) the local secondary structure. Based on these parameters, each residue position is categorized into an environment class. In this manner, a three-dimensional protein structure is converted into a one-dimensional environment string, which represents the environment class of each residue in the folded protein structure. A 3D structure profile table is then created containing score values that represent the frequency of finding any of the 20 common amino acids structures at each position of the environment string. These frequencies are determined from a database of known protein structures and aligned sequences. The method determines the most favorable alignment of a target protein sequence to the residue positions defined by the environment string, and determines a “best fit” alignment score, Sij for the target sequence. Each target sequence may then be further characterized by a ZScore, which is the number of standard deviations that Sij for the target sequence is above the mean alignment score for other target sequences of similar length.” International Patent Application No. WO 93/01484 is incorporated into this application by reference.

International Patent Application No. WO 98/48270 published Oct. 29, 1998 for a method of determining three-dimensional protein structure from primary protein sequence by William Goddard et al. assigned to the California Institute of Technology provides the following background information, “The Generic Protein method is a computer-implemented system for determining the three-dimensional structure of a protein from its amino acid sequence. The method incorporates a hierarchical approach wherein the number of candidate structures decreases at each step. The starting point is the use of a sequence independent ensemble of compact structures which represents an exhaustive enumeration of all possible self-avoiding folded topologies for an residue polypeptide. Because the number of candidate conformations is dramatically reduced, recognition filters such as radius of gyration, distribution of hydrophobic residues, and the satisfaction of disulfide constraints can be used to further reduce the number of candidate conformations. The complexity of the initial ab initio structure prediction problem can be reduced to a complexity on the order of a homology modeling exercise. The final refinement step may involve molecular mechanics procedures with explicit solvation parameters on full-atom representations of the remaining candidate structures.” International Patent Application No. WO 98/48270 is incorporated into this application by reference.

International Patent Application No. WO 00/11206 published Mar. 2, 2000 for methods and systems for predicting protein function by Jeffrey Skolnick et al. assigned to the Scripts Research Institute provides the following background information, “The present invention concerns methods and systems for predicting the biological function(s) of proteins. The invention is based on the development of functional site descriptors for discrete protein biological functions. Functional site descriptors are geometric representations of protein functional sites in three-dimensional space, and can also include additional parameters, for example, conformational information. Following their development, one or more functional site descriptors (for one or more different biological functions) are used to probe protein structures to determine if such structures contain the functional sites described by the corresponding functional site descriptors. If so, the protein(s) containing the functional site(s) are predicted to have the corresponding biological function(s). In preferred embodiments, a library of functional site descriptors is used to probe inexact protein structures derived by computational methods from amino acid sequence information to predict the biological function(s) of such sequences and of the gene(s) encoding the same.” International Patent Application No. WO 00/11206 is incorporated into this application by reference.

### SUMMARY

Features and advantages of the present invention will become apparent from the following description. Applicants are providing this description, which includes drawings and examples of a specific embodiment, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Embodiment of the present invention provides a method of producing information about proteins and apparatus for producing information about proteins. The method comprises a number of steps. The embodiment includes the following steps: Developing a specification of how local descriptors of protein structure are defined in the protein structure. Developing a local descriptors of protein structure database including calculated descriptors. Developing a definition of inter-descriptor similarity measure. Developing a local descriptors of protein structure class database of descriptors organized by their similarity. Producing information about any protein by analyzing said protein using the local descriptors of protein structure class database of descriptors organized by their similarity.

### BRIEF DESCRIPTION OF THE DRAWINGS

The accompanying drawings, which are incorporated into and constitute a part of the specification, illustrate specific embodiment of the invention and, together with the general description of the invention given above, and the detailed description of the specific embodiment, serve to explain the principles of the invention.

FIG. 1 illustrates a system for producing information about a protein.

FIG. 2 shows the C_{βx} position.

FIG. 3 shows a descriptor 1ayl_A #459 is formed by 9 elements and consisting of 5 segments.

FIG. 4 is the number of domains from the ASTRAL 1.57 database presented as a bar diagram in terms of a number of descriptors found in each domain of Applicants database.

FIG. 5 is a bar diagram of the number of descriptors in Applicants database in terms of the number of segments in each descriptor.

FIG. 6 is a graph showing the number of groups by the number of main chain segments they comprise; only groups comprising three and more segments are shown.

FIG. 7 is an example wherein a notation is introduced to classify descriptor groups by their secondary structure.

FIG. 8 illustrates the reduction in the descriptor group dataset when a process of in-category redundancy reduction is performed.

FIG. 9 shows the statistics for the intra- and inter-category redundancy elimination procedure.

FIG. 10 describes global coverage statistics for the entire database of protein structures.

FIG. 11 shows mean and median values of LDPS class population depending on number of segments in LDPS class.

FIG. 12 shows mean and median values of number of different folds in LDPS classes.

FIG. 13 shows the sequence location of the 57 identified LDPS classes

FIG. 14 shows sequence of the same protein annotated with levels of coverage in terms of LDPS classes.

### DETAILED DESCRIPTION OF THE INVENTION

Referring now to the drawings, to the following detailed information, and to incorporated materials; a detailed description of the invention, including a specific embodiment, is presented. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

It is widely accepted that a protein's amino acid sequence uniquely encodes the final structure, so that in principle the structure should be predictable from sequence alone. In practice, the subtlety and vast size of the protein folding energy landscape means that truly first principle prediction of structure is still elusive. However, in recent years, dramatic developments in genome sequencing and structure determination have greatly expanded the scope of the homology based prediction methods. With the knowledge of large sequence families, often from several species, and the increasing number of structures solved, these methods can be used to model a significant fraction of protein structures. The basis is the strong conservation of protein three-dimensional fold across large evolutionary distances, within species and across species, irrespective of sequence variation. It is estimated that the number of proteins for which it is possible to obtain structure models by homology is 1-2 orders of magnitude greater than the number of experimentally determined structures. In large-scale whole genome applications, approximately 40% of the open reading frames can be at least in part characterized structurally. Thus, with the approximately 700,000 protein sequences already known (approximate number of databank entries less than 90% identical), an estimated 280,000 could be modeled. This number compares with the approximately 2,000 distinct structures deposited in the PDB (out of a total of 19,000). By providing additional modeling templates, the current initiatives in experimental structure determination will even further increase the number of structures that could be modeled.

Although experimental methods for determining protein structure, primarily X-ray crystallography and NMR spectroscopy, have advanced considerably in the last few years, they are presently providing structures more than 100 times more slowly than they are being sequenced. Instead, it is expected that in the near future the automated protein structure prediction methods will make significant impact on the impending onerous task of analyzing the large number of unknown protein sequences generated by the ongoing genome-sequencing projects. Automated methods, which will allow assigning or solving the protein structure computationally have a very significant practical value. Invention described in this application provides means to define, organize, and analyze the relationships between protein sequence and structure found by experiment and available via Protein Databank, the public database of protein structures.

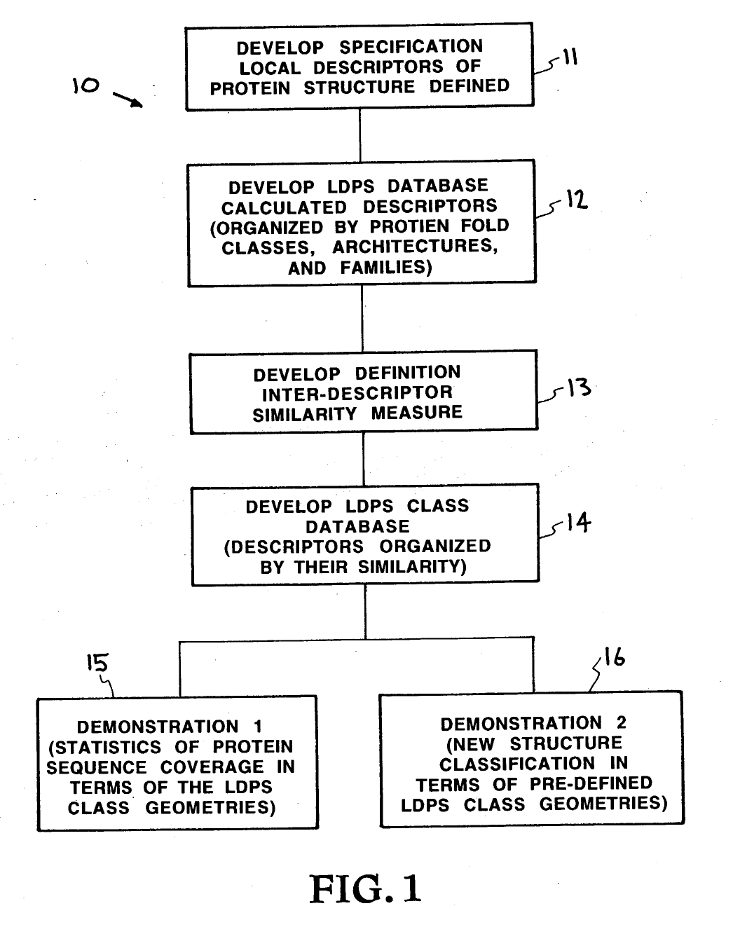

Referring now to FIG. 1, a system for producing information about a protein is illustrated. The system is designated generally by the reference numeral 10. The main task of the system is to identify a set of relationships between sequence and structure allowing a structural characterization of a new (query) sequence. It is assumed that novel, yet uncharacterized, protein structures are built from the same repertoire of small local structure building blocks hereinafter called “descriptors” or “local descriptors of protein structure (LDPS).” Local sequences associated with each class of descriptors are generalized to identify known local structures among new protein sequences, leading to the structural characterization of proteins for which little or no structural data are available.

A specification of how local descriptors of protein structure are defined for amino acids in the protein structure is developed, a local descriptors of protein structure database including calculated descriptors is developed, a definition of inter-descriptor similarity measure is developed, a local descriptors of protein structure class database of descriptors organized by their similarity is developed, and information about the protein is produced by analyzing the protein using the local descriptors of protein structure class database of descriptors organized by their similarity.

The steps system 10 can be summarized as follows. Step 11 comprises developing specifications of how local descriptors of protein structure are defined for each of the amino acids in a protein structure. Step 12 comprises developing local descriptors of protein structure database of all calculated descriptors, organized by protein fold classes, architectures, and families. Step 13 comprises developing definitions of inter-descriptor similarity measure. Step 14 comprises developing local descriptors of protein structure class database of descriptors organized by their similarity. Steps 15 and 16 comprise Demonstration 1 and Demonstration 2. Demonstration 1, designated by the reference numeral 15, comprises developing statistics of protein sequence coverage in terms of the local descriptors of protein structure class geometries. Demonstration 2, designated by the reference numeral 16, comprises developing new structure classification in terms of pre-defined local descriptors of protein structure class geometries.

The system 10 has numerous uses. For example, the system 10 can be used for analysis of protein structure, superposition of protein structures and their subsets, comparative (homology) modeling of proteins, protein fold recognition, structural and functional annotation of genomes, and ab initio prediction of protein structure.

The steps of the system 10 will now be described in greater detail. Applicants have established that adequate database representation exists for the tightly packed regions of protein structure, sufficient to fully describe each known protein fold, and suggesting that novel, yet uncharacterized, protein structures are built from the same repertoire of small local structure building blocks Applicants call descriptors. Applicants have also established that the sequences associated with each class of descriptors can be generalized to identify known local structures among new protein sequences, leading to the structural characterization of proteins for which little or no structural data are available.

The Step 11 generates a specification of local descriptors of protein structure. The purpose of Step 11 is to generate a database of local descriptors of protein structure. Environmental description of each residue is used to build a new type of database of all proteins from the PDB Data Bank. The software is written in C language.

In general, a descriptor encompasses a few segments of a protein chain localized near the selected amino acid residue. In case when several chains of the same protein come close to each other in space and can be tracked back to a single genetic source forming a so-called “genetic domain,” descriptor can encompass segments belonging to different chains.

It is therefore tied to a particular amino acid, with its number reflected in the descriptor's name. Applicants use two naming schemes to identify descriptors. If Applicants analyze protein chains as they are represented in the PDB database (see http://www.rcsb.org/pdb) Applicants use the PDB ID of the protein followed by an underscore and a chain name (if any) and the residue number associated with the descriptor, e.g. 1ayl_A#459 or 1chd#278. If Applicants use the ASTRAL database (see Chandonia J M, Walker N S, Lo Conte L, Koehl P, Levitt M, Brenner SE. ASTRAL compendium enhancements. Nucleic Acids Research 30:260-263 (2002)) of SCOP-derived protein domains Applicants use the ASTRAL-type domain name followed by the residue number, e.g. 1e43a2#231 (protein 1e43, chain a, domain 2, residue 231) or 1h8d.1#H221A (protein 1h8d, genetic domain 1, residue 221A from chain H).

The basic element used to build a descriptor is a 5 residue-long fragment of protein backbone. To build a descriptor out of such fragments for a particular residue, Applicants check distances between the selected residue and all other residues in the protein and, if they are close in space, Applicants include that residue together with its four neighbors (two residues on both sides of that residue in the protein's amino acid sequence).

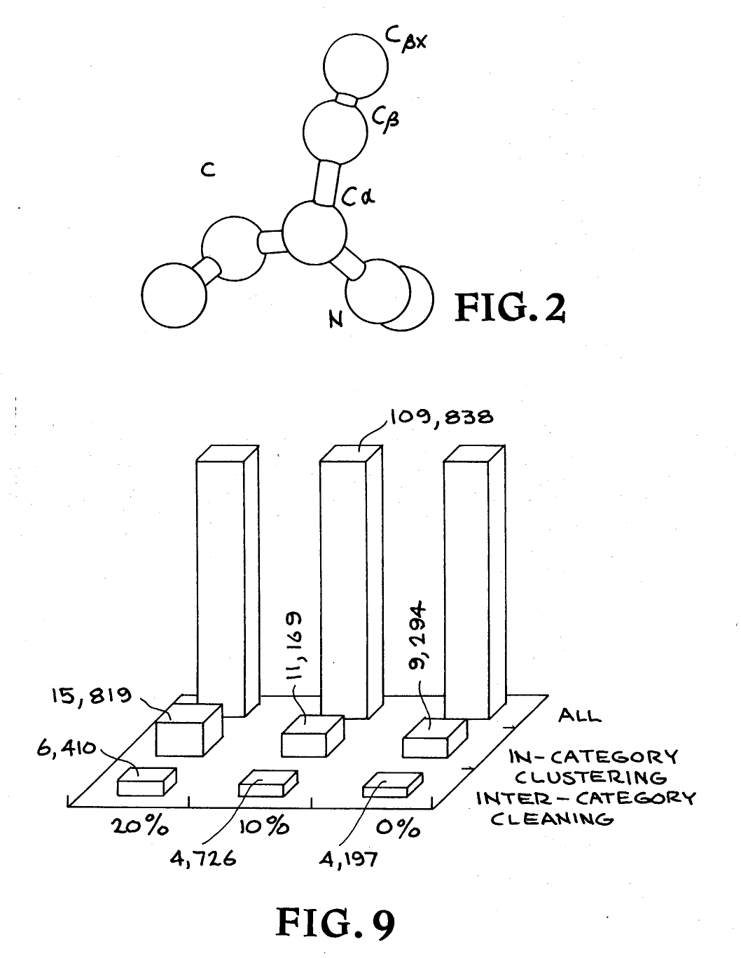

Defining distance between residues Applicants imply that protein residues can be considered as material points. To implement this approximation Applicants set the center of each residue along the extension of the C_{α}→C_{β} vector at the distance of 2.5 Å from the C_{α} a atom, as it is shown in FIG. 2 (for glycine Applicants define this point based on a regular amino acid geometry and using the N, C_{α}, and C atoms). FIG. 2 shows C_{βx} position. In so doing Applicants put the C_{β} atom approximately in the center of a typical residue and implicitly introduce the presence of a side chain into subsequent calculations, which is important due to the fact that a large part of residue mass is usually localized in its side chain. This definition allows Applicants to use the same, relative to the backbone conformation, position in space to identify geometrically different residues.

In Applicants definition two residues identified by numbers i and j are considered to be close to one another in space if the Euclidian distance between their C_{βxS} satisfies one of the two following conditions:

|C_{βx}⁽ⁱ⁾−C_{βx}^{(j)}|<6.5 Å or

6.5 Å<|C_{βx}⁽ⁱ⁾−C_{βx}^{(j)}|<8 Å and |C_{βx}⁽ⁱ⁾−C_{βx}^{(j)}|<|C_{α}⁽ⁱ⁾−C_{α}^{(j)}|−0.75 Å.

These parameters were set empirically to reach a balance between requirements of a small size and of a sufficient structural specificity of a descriptor.

Such defined elements are checked for overlap, in case of which they are joined into segments. For example, if two elements consisting of residues 454-458 and 457-461 are found in the same descriptor, they are concatenated into one segment consisting of residues 454-461. Thus, each segment is at least 5 residues long and may consist of one or several elements. The segment encompassing the central element, i.e. containing the descriptor-defining residue, is called the main segment.

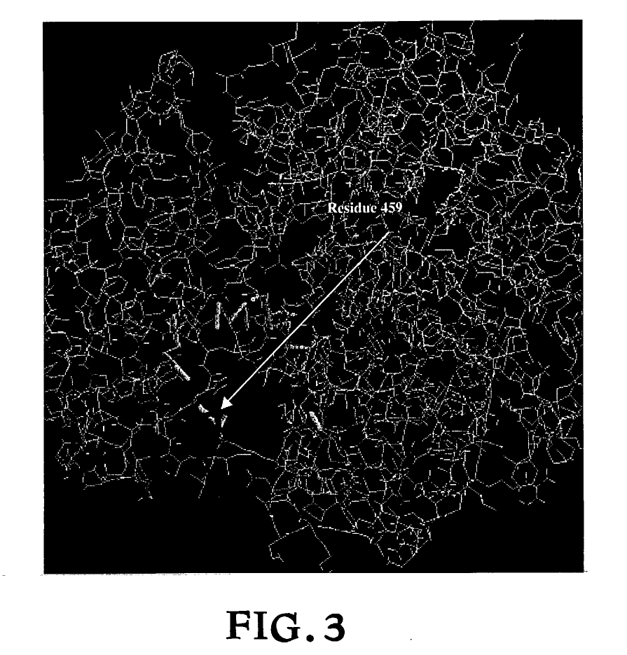

Referring now to FIG. 3, a descriptor 1ayl_A #459 is formed by 9 elements and consists of 5 segments. In the example shown in FIG. 3, the number of the central residue is 459 and therefore the central element consists of residues 457-461 and the main segment is the one encompassing residues 454-465 in this case. Collection of all such defined segments forms a descriptor associated with the central residue. Note, that there may be n-4 descriptors in each protein chain, where n is the number of amino acids in that chain.

The Step 12 generates local descriptors of protein structure database of all calculated descriptors, organized by protein fold classes, architectures, and families. Local descriptors of protein structure are calculated for a non-redundant set of protein structures, thus limiting similarities necessitated by any significant sequence homology. Applicants restrict the calculation of structural similarity to a data set of structures with sequence identity not greater than 40% (ASTRAL—Chandonia J. M., Walker N. S., Lo Conte L., Koehl P., Levitt M., Brenner S. E. ASTRAL compendium enhancements. Nucleic Acids Research 30 (2002), 260-263—database). This database contains a subset of SCOP domains for which no two domains have more than 40% sequence identity. Its 1.57 release—Chandonia J. M., Walker N. S., Lo Conte L., Koehl P., Levitt M., Brenner S. E. ASTRAL compendium enhancements. Nucleic Acids Research 30 (2002), 260-263—(March 2002) contains 4013 entries. Working with this database, Applicants are able to build 714,496 descriptors, approximately 178 descriptors per protein chain on the average. Average number of elements in a descriptor is approximately 6.5. The longest domain, d1i50a_, contains 1419 residues.

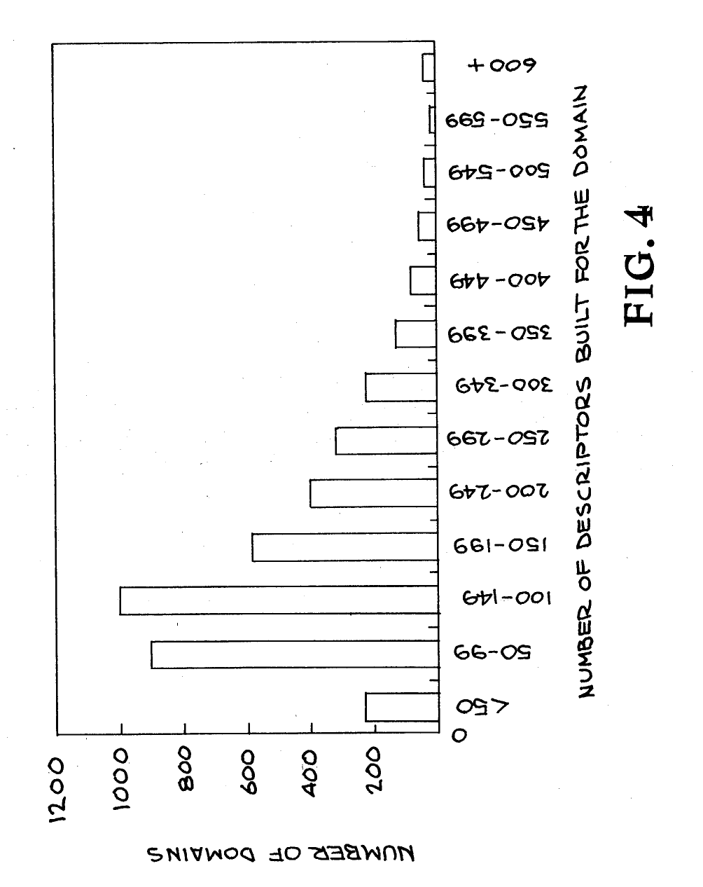

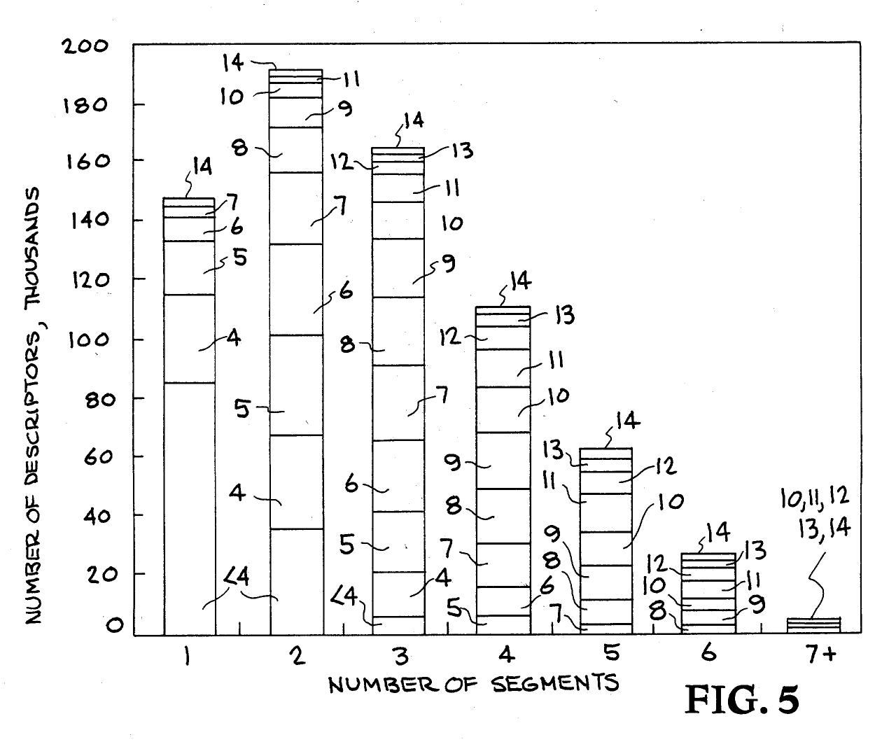

The charts, FIGS. 4 and 5, show the distributions of the number of descriptors per protein domain and of the number of descriptors of a specific size in terms of the number of segments. FIG. 4 shows the number of domains from the ASTRAL 1.57 presented as a bar diagram in terms of a number of descriptors found in each domain of Applicants database. FIG. 5 is a bar diagram of the number of descriptors in Applicants database in terms of the number of segments in each descriptor. The corresponding number of elements is also shown by color-coding.

The Step 13 develops definition of an inter-descriptor similarity measure. To compare descriptors Applicants have developed a multilevel structural similarity function. This function encompasses:

number of segments,

number of elements,

geometry of the central element,

number of pairs (central element+any other element) from both descriptors that are similar under the specified RMSD cutoff, and

overall RMSD score between descriptors.

More specifically, Applicants first compare the number of segments and elements in the two descriptors. Two descriptors pass the first requirement if they fulfill the following two inequalities:

1/T₁<m₁/m₂<T₁

and

1/T₂<n₁/n₂<T₂,

where mᵢ and nᵢ (i=1,2) are numbers of respectively segments and elements in the i^{th} descriptor, Tₖ (Tₖ>1, k=1,2) are predefined constants. Note, that the second condition implicitly disallows descriptors with substantially different length of segments to be considered as similar. If these necessary but not sufficient similarity conditions are not satisfied then the two descriptors are considered to be different and Applicants stop here.

Second, Applicants proceed to verify if the geometries of the central elements do not differ significantly. At this point Applicants superimpose geometrical shapes of the two specified 5-residue long C_{α} traces of the protein backbones as well as possible. To evaluate the dissimilarity between geometrical conformations of these elements Applicants use the root mean squared distance measure (RMSD). In other words, Applicants are superimposing two 5-point vectors (each point represents a residue in the central element) and minimizing the distance function. The minimum value of this function, is required to satisfy the following condition:

RMSD (0₁, 0₂)<T₃

where 0ᵢ (i=1,2) denotes the central element of the i^{th} descriptor and T₃ is an RMSD cutoff.

Third, descriptor structures are compared as a whole. A combinatorial problem has to be solved to determine which element in one descriptor corresponds with which in the other one to obtain the best fitness. Statistics show that the average number of elements in descriptors containing not fewer than 3 segments is approximately 8.5. Therefore for each pair of descriptors Applicants must check Γ (8.5)≈14,000 variants to find the best fitness. (Γ is Euler's gamma function) In order to speed up the process Applicants have developed an approach that allows them to compare descriptors making comparisons of the kind “all elements against all” only rarely. To achieve this Applicants consider the central element together with all the other elements jᵢ (i=1,2) of this descriptor one by one (jᵢ=1ᵢ, . . . , nᵢ) as a stiff system (note that nᵢ, i=1,2, is the number of elements in the i^{th} compared descriptor). Applicants then compare this 10 point vector from the first descriptor with all possible 10 point vectors from the second descriptor for fitness under a specified RMSD cutoff:

RMSD (0₁∪j₁, 0₂∪j₂)<T₄

Note, that by this procedure Applicants acquire knowledge as to which elements in the compared descriptors correspond to one another. And, as a result, Applicants obtain one to one structural alignment between residues in similar descriptors.

The Step 14 develops local descriptors of protein structure class database of descriptors organized by their similarity. It generates a database of descriptor classes. For a specified residue in a protein chain and the corresponding descriptor called the “seed descriptor” the program identifies and catalogues all other similar sub-structures found in a non-redundant database of protein structures (a subset of Protein Databank). (In a specific application the program compares pairs of protein structures by evaluating the similarity between residue-based descriptors.) The software is written in C language.

Populating a database of descriptor classes proceeds in three steps. First Applicants generate highly redundant descriptor groups by applying the similarity criteria described in the previous section for a specified set of parameters Tₖ. This procedure is dependent on the value of parameters Tₖ. For example, choosing T₁=T₂=2, T₃=1.3 Å, T₄=1.5 Å generates 109,838 descriptor groups of at least seven examples in each group.

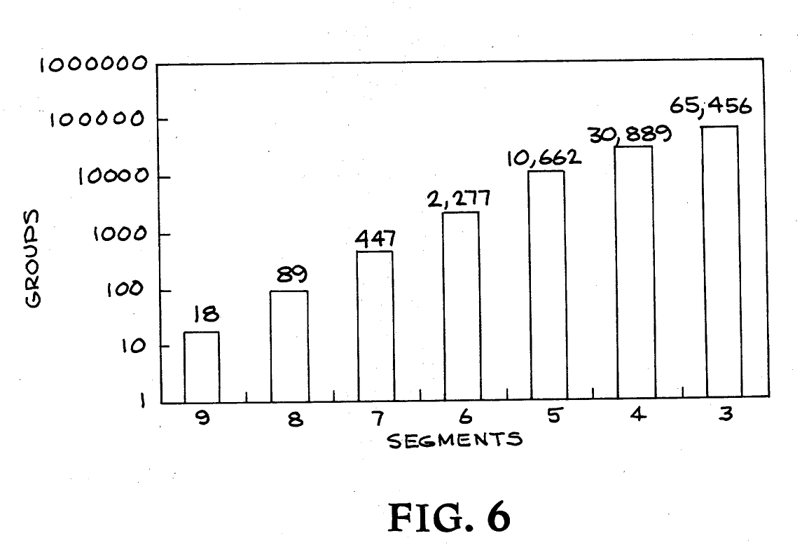

FIG. 6 illustrates the number of groups by the number of main chain segments they comprise. The number of groups increases (y-axis is scaled logarithmically) with decreasing number of segments they contain.

Second, Applicants check the number and the secondary structure of segments in each group and organize groups by the type of secondary structure of the main chain segments they comprise. To assign a specific secondary structure (SS) tag to a segment as a whole Applicants use the DSSP (Brenner S. E., Koehl P., Levitt M. The ASTRAL compendium for protein structure and sequence analysis, Nucleic Acids Research 28 (2000), 254-256) program and assign SS tag to each residue in the segment. Applicants assume that residues that are identified by DSSP as H, G, I are helices (h), as E and B are β-strands (e) and all others are coil (c). If more than 50% of residues in the segment are identified as helices and less than 20% are identified as β-strands then the whole segment is identified as helical. Vise versa, Applicants assign β-strand structure to a segment if more than 50% of residues are identified as β-strands and less than 20% as helices. In all other cases (e.g. for the segment with the following SS structure: eeecchh) or when more than 50% of residues are being identified as coil, Applicants assign a coil tag to the segment.

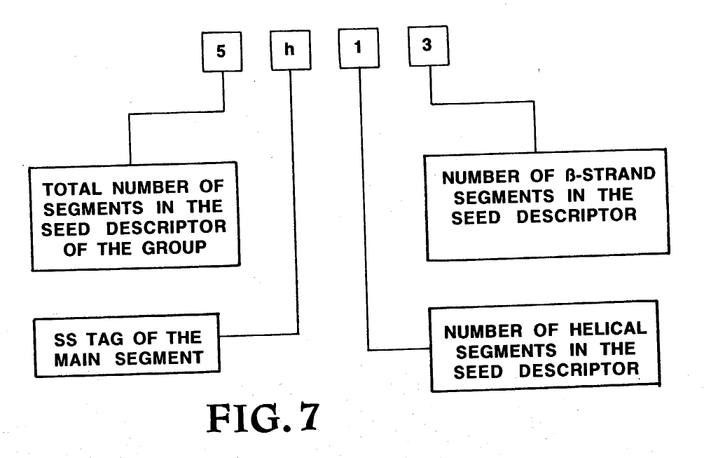

Referring now to FIG. 7, a notation is introduced to classify descriptor groups by their secondary structure. The notation “5h13” includes the following: “5”-total number of segments in the seed descriptor of the group, “h”-SS tag of the main segment, “1”-number of helical segments in the seed descriptor, and “3”-number of β-strand segments in the seed descriptor. In the example, the number of helical segments taken together with the number of β-strand segments does not sum up to the total number of segments in the descriptor: the resulting difference is the number of segments classified as coil.

Third, Applicants remove redundancy from group classification. This step is independent from secondary structure classification and has a primary goal of generating a small number of distinct descriptor classes. In most cases each resulting descriptor class will have assigned a single secondary structure category. Applicants utilize this observation to reduce the number of necessary comparisons. Applicants proceed by performing a series of pair-wise descriptor group comparisons and requiring that no more than n% of descriptors in the smaller of the two groups are identical with descriptors in the larger group.

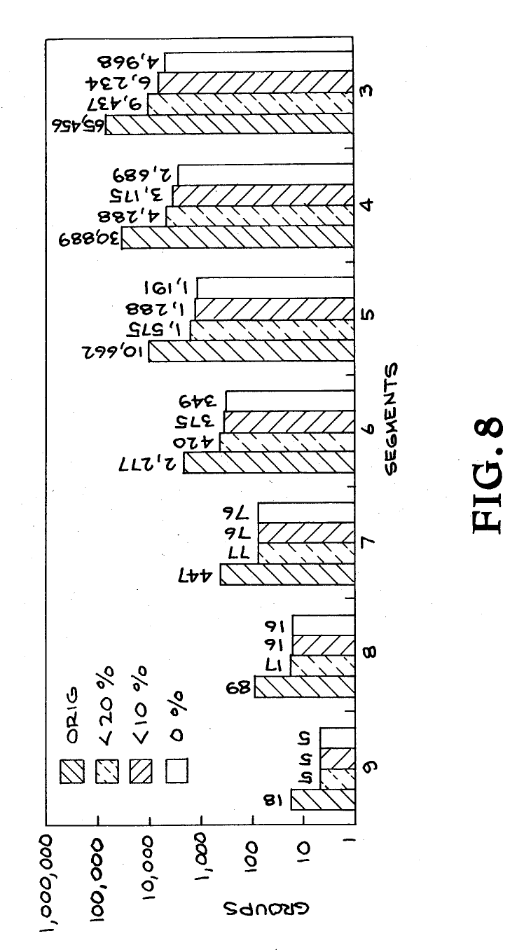

In FIG. 8 Applicants illustrate the reduction in the descriptor group dataset as the process of intra-category redundancy reduction is performed. Violet bars correspond to the initial number of groups in classes; blue bars show reduction in the number of distinct groups after Applicants apply the 20% common descriptors criterion; red bars—10% of common descriptors criterion; and yellow bars—no common descriptors criterion. The resulting number of groups is just over 9,000 at the 0% redundancy level.

In the second part of redundancy elimination Applicants perform inter-category comparisons. FIG. 9 shows a comparison of the number of groups after both intra- and inter-category steps of the clustering process for the three levels of allowed redundancy. It shows the statistics for redundancy elimination procedure performed at 20, 10, and 0% levels.

The Step 15 provides a demonstration 1, statistics of protein sequence coverage in terms of the local descriptors of protein structure class geometries. Applicants find that the local descriptors of protein structure (LDPS) classes bridge proteins not related by sequence homology and are sufficiently common to allow the characterization of new folds. In some cases local geometries found on protein surfaces are unique to a given structure but such regions are limited and do not detract from formalism's ability to describe protein folds. Cores of protein structures exhibit less variability than surfaces, producing much greater sampling. Thus, when both of these two types of data are considered, the current protein structure database (the PDB) can be assessed to contain a sufficient number of LDPS classes to describe practically all new protein structures.

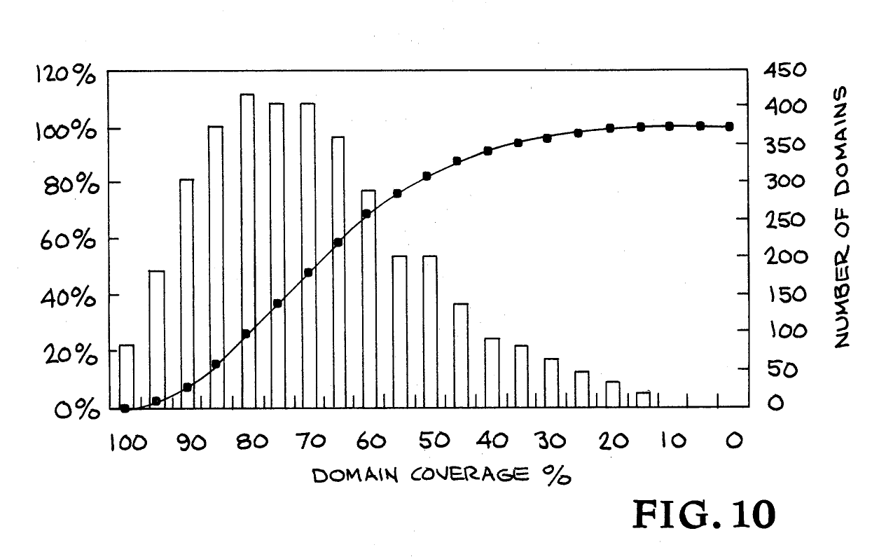

FIG. 10 describes global coverage statistics for the entire database of protein structures (shown as structural domains) in terms of the LDPS classes of three or more main chain segments and 7 or more descriptors within a class. FIG. 10 describes LDPS class coverage of protein structural domains in a non-redundant database. One set of bars represent the number of domains binned by the degree of coverage. The set of squares correspond to a cumulative percent of all domains as a function of coverage. For example, the most common domain coverage is found in the 70-80% region, while more than 90% of all domain sequences are more than 40% covered.

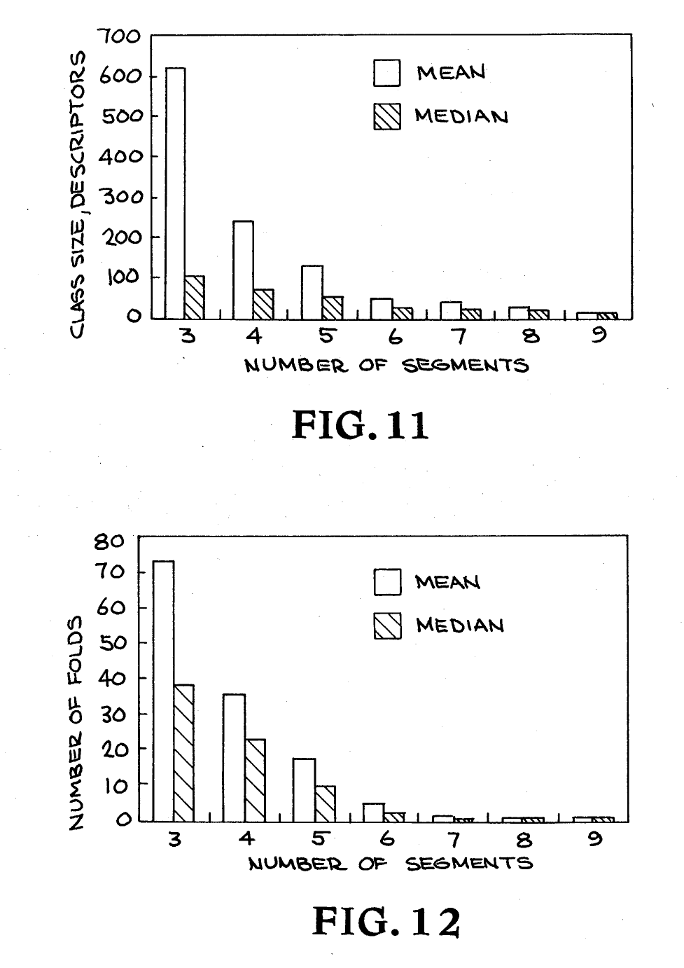

FIG. 11 shows mean and median values of LDPS class population depending on number of segments in LDPS class. Class population varies greatly with the number of segments comprised by classes, rapidly decreasing for classes containing larger number of segments. Note that it is necessary to take into account both mean and median values of population in the LDPS classes as deviation as skewness in population distribution rapidly increases with decreasing number of segments in a class.

FIG. 12 shows mean and median values of number of different folds in LDPS classes comprising descriptors with different numbers of segments. It is also interesting to ask in how many different protein folds a given LDPS class can be found. The analysis shows that only descriptors with a large number of segments are fold specific. For example, all LDPS classes with 8 or 9 segments and the majority of classes with 7 segments refer to only one fold, while classes with 3- or 4-segments can be found in large number of folds.

The Step 16 provides a demonstration 2, new structure classification in terms of pre-defined local descriptors of protein structure class geometries. To demonstrate the LDPS formalism's ability to characterize a protein structure Applicants have performed the calculation for a randomly selected protein domain 1qgo (chain A). In agreement with the general finding of the first demonstration, the sequence of the test protein is sufficiently covered with a total of 57 LDPS classes.

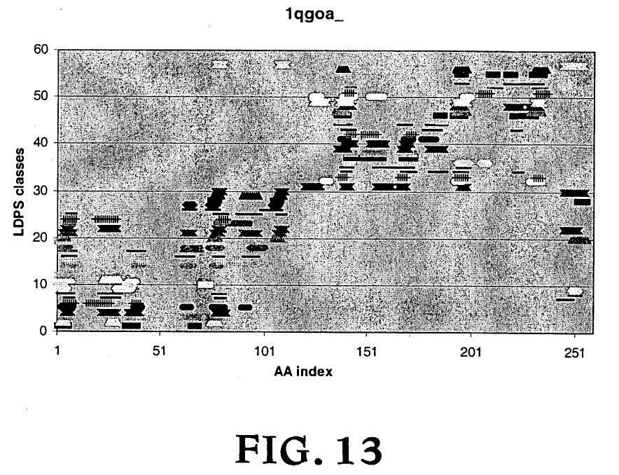

FIG. 13 illustrates coverage of the test protein (1qgo chain A) by LDPS classes. Each class is marked by a different symbol and color and is plotted on the same level, corresponding to its consecutive number (a total of 57 LDPS classes are plotted; the greater the AA number to which a given LDPS class is assigned the higher along the Y-axis it is plotted).

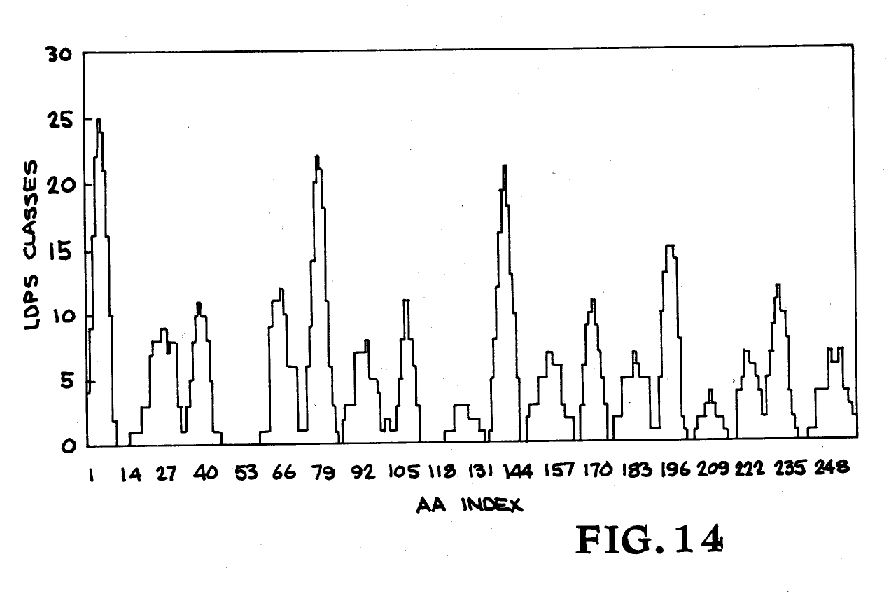

FIG. 14 shows sequence of the same protein (1qgo chain A) annotated with levels of coverage in terms of LDPS classes. Levels of coverage vary greatly reflecting the degree of popularity a local geometry associated with a particular amino acid has among other known protein structures. Peaks correspond to locations inside protein core while troughs to the surface of the molecule, where local structure is less typical or is unique.

Several potential applications of the local descriptors, all requiring additional methods and software development were developed as follows:

(A) Structure superposition. Superposition techniques lie at the heart of meaningful comparisons of protein structures. For the sake of clarity, two separate modes of structure superposition can be described: sequence-dependent and sequence-independent. The sequence-dependent superposition aligns protein residues in a 1:1 correspondence, according to a specified sequence alignment. The sequence-independent superposition, on the other hand, is not restricted by the 1:1 correspondence requirement and is capable of identifying regions of similarity regardless of the sequence alignment. Sequence-independent superpositions present a computationally difficult problem and even the best procedures developed to date do not guarantee finding the optimal superposition. Both the sequence-dependent and sequence-independent structural alignments are typically of the rigid-body type. Rigid-body superpositions, when not carefully applied, are likely to produce misleading results in the case of multi-domain structures, where one domain is shifted relative to another. They will also perform poorly when similarity between structures is characterized by a gradual deformation of one relative to the other. Application of the descriptor formalism to the problem of structure superposition, allows implementing an algorithm capable of sequence-independent, non-rigid-body type superpositions.

(B) Comparative (homology) modeling. Comparative modeling relies on the fact that for all pairs of natural proteins so far encountered, a clear sequence homology implies similar structures. Thus, the structure of a homologous protein (template) can to a large degree guide generating a model of a new one. The most error-prone in this process are the alignments between the target sequence and the template structure. The sequence signal derived from similar segments of protein structure for each of the considered residues in the template will provide an independent evaluation of a putative sequence-to-structure alignment. Application of the descriptor formalism in this case thus helps eliminate the major source of errors in comparative modeling.

(C) Fold recognition (threading). Fold recognition takes advantage of the fact that protein structure is much more strongly conserved than sequence. This means that typically many protein sequences adopt a similar fold. The goal is to identify these structural relationships in cases where sequence signal is either weak or does not exist. The descriptor-derived sequence signal, a signal independent from protein family considerations or knowledge of close sequence homologues, allows assigning protein folds in such cases.

(D) Ab initio structure prediction. This is the most challenging application, as the sequence signal associated with the library descriptors is used to identify the most remote homologues. In this approach there are no template structures to facilitate the verification of the structural hypothesis. The verification is thus based on structural consistency of the assignments rather than on the consistency with a library template (as in the case of fold recognition). However, even with partial success of this application, descriptors contribute to solving a major scientific and practical problem in biology.

It should be understood that the invention is not intended to be limited to the particular forms disclosed. Rather, the invention is to cover all modifications, equivalents, and alternatives falling within the spirit and scope of the invention as defined by the following appended claims.

The invention claimed is

1. A method of producing information about a protein using a protein structure, comprising the steps of:
developing a specification of how local descriptors of protein structure are defined,
developing a local descriptors of protein structure database including calculated descriptors,
developing a definition of inter-descriptor similarity measure,
developing a local descriptors of protein structure class database of descriptors organized by their similarity, and
producing information about any protein by analyzing said protein using the local descriptors of protein structure class database of descriptors organized by their similarity.

2. The method of producing information about a protein of claim 1, wherein said step of producing information about said protein by analyzing said protein using said local descriptors of protein structure class database of descriptors organized by their similarity, comprises analyzing said protein on the basis of statistics of protein sequence coverage in terms of local descriptors of protein structure class geometries.

3. The method of producing information about a protein of claim 1, wherein said step of producing information about said protein by analyzing said protein using said local descriptors of protein structure class database of descriptors organized by their similarity, comprises analyzing said protein on the basis of new structure classification in terms of pre-defined local descriptors of protein structure class geometries.

4. The method of producing information about a protein of claim 1, wherein said step of developing a local descriptors of protein structure database including calculated descriptors is organized by protein fold classes, architectures, and families.

5. The method of producing information about a protein of claim 1, wherein said step of developing a local descriptors of protein structure class database of descriptors organized by their similarity is completed using said definition of inter-descriptor similarity measure.

6. The method of producing information about a protein of claim 1, wherein said step of producing information about said protein by analyzing said protein using said local descriptors of protein structure class database of descriptors organized by their similarity provides information relating to analysis of protein structure.

7. The method of producing information about a protein of claim 1, wherein said step of producing information about said protein by analyzing said protein using said local descriptors of protein structure class database of descriptors organized by their similarity provides information relating to superposition of protein structures and their subsets.

8. The method of producing information about a protein of claim 1, wherein said step of producing information about said protein by analyzing said protein using said local descriptors of protein structure class database of descriptors organized by their similarity provides information relating to comparative (homology) modeling of proteins.

9. The method of producing information about a protein of claim 1, wherein said step of producing information about said protein by analyzing said protein using said local descriptors of protein structure class database of descriptors organized by their similarity provides information relating to protein fold recognition.

10. The method of producing information about a protein of claim 1, wherein said step of producing information about said protein by analyzing said protein using said local descriptors of protein structure class database of descriptors organized by their similarity provides information relating to structural and functional annotation of genomes.

11. The method of producing information about a protein of claim 1, wherein said step of producing information about said protein by analyzing said protein using said local descriptors of protein structure class database of descriptors organized by their similarity provides information relating to ab initio prediction of protein structure.

12. A system for producing information about a protein using a protein sequence, comprising:
means for developing a specification of how local descriptors of protein structure are defined in said protein sequence,
means for developing a local descriptors of protein structure database including calculated descriptors,
means for developing a definition of inter-descriptor similarity measure,
means for developing a local descriptors of protein structure class database of descriptors organized by their similarity, and
means for producing information about said protein by analyzing said protein using the local descriptors of protein structure class database of descriptors organized by their similarity.

13. The system for producing information about a protein of claim 12, wherein said means for producing information about said protein by analyzing said protein using said local descriptors of protein structure class database of descriptors organized by their similarity, comprises a means for analyzing said protein on the basis of statistics of protein sequence coverage in terms of local descriptors of protein structure class geometries.

14. The system for producing information about a protein of claim 12, wherein said means for producing information about said protein by analyzing said protein using said local descriptors of protein structure class database of descriptors organized by their similarity, comprises a means for analyzing said protein on the basis of new structure classification in terms of pre-defined local descriptors of protein structure class geometries.

15. The system for producing information about a protein of claim 12, wherein said means for developing a local descriptors of protein structure database including calculated descriptors is organized by protein fold classes, architectures, and families.

16. The system for producing information about a protein of claim 12, wherein said means for developing a local descriptors of protein structure class database of descriptors organized by their similarity is completed using said definition of inter-descriptor similarity measure.

17. The system for producing information about a protein of claim 12, wherein said means for producing information about said protein by analyzing said protein using said local descriptors of protein structure class database of descriptors organized by their similarity provides information relating to analysis of protein structure.

18. The system for producing information about a protein of claim 12, wherein said means for producing information about said protein by analyzing said protein using said local descriptors of protein structure class database of descriptors organized by their similarity provides information relating to superposition of protein structures and their subsets.

19. The system for producing information about a protein of claim 12, wherein said means for producing information about said protein by analyzing said protein using said local descriptors of protein structure class database of descriptors organized by their similarity provides information relating to comparative (homology) modeling of proteins.

20. The system for producing information about a protein of claim 12, wherein said means for producing information about said protein by analyzing said protein using said local descriptors of protein structure class database of descriptors organized by their similarity provides information relating to protein fold recognition.

21. The system for producing information about a protein of claim 12, wherein said means for producing information about said protein by analyzing said protein using said local descriptors of protein structure class database of descriptors organized by their similarity provides information relating to structural and functional annotation of genomes.

22. The system for producing information about a protein of claim 12, wherein said means for producing information about said protein by analyzing said protein using said local descriptors of protein structure class database of descriptors organized by their similarity provides information relating to ab initio prediction of protein structure.

23. The system for producing information about a protein of claim 12, including means for predicting the structure of said protein.
